(19) United States
(12) Patent Application Publication
(10) Pub. No.: US 2016/0176961 A1
(43) Pub. Date: Jun. 23, 2016

(54) THROMBOPOIETIN RECEPTOR LIGANDS FOR NEUROPROTECTION

(71) Applicant: MCW Research Foundation, Inc., Milwaukee, WI (US)

(72) Inventor: John E. Baker, Wauwatosa, WI (US)

(21) Appl. No.: 15/054,935

(22) Filed: Feb. 26, 2016

Related U.S. Application Data
(62) Division of application No. 14/348,147, filed on Jul. 16, 2014.

(51) Int. Cl. C07K 16/26; A61K 9/00; A61K 38/22; A61K 31/4152

(52) U.S. Cl. CPC C07K 16/26; A61K 31/4152; A61K 9/0019; A61K 38/22; C07K 2317/41

(57) ABSTRACT

Methods for the treatment of an ischemic event (e.g., stroke) that creates or embodies a risk of neurological damage in CNS sites by administration of a thrombopoietin receptor ligand.

### CROSS-REFERENCE TO RELATED APPLICATIONS

This application is a divisional of U.S. Ser. No. 14/348,147 which is a 35 U.S.C. §371 National Stage Application of International Application No. PCT/US2011/053904, filed Sep. 29, 2011, the entire contents of which are incorporated herein by reference.

### SEQUENCE LISTING

The specification includes a Sequence Listing in the form of an ASCII compliant text file named “2010044-0012_ST25”, which was created on Feb. 26, 2016 and has a size of 9,242 bytes, the content of which is incorporated herein by reference.

### BACKGROUND

In the United States, strokes strikes 700,000 people annually. Of these stroke patients, 40% will die, the equivalent of one death every three minutes. Fifty percent of stroke-related deaths occur in the hospital. These facts indicate an urgent need to develop a treatment to reduce mortality and morbidity arising from stroke.

### SUMMARY

The present invention encompasses the insight that thrombopoietin receptor ligands are useful agents for use in the treatment of an ischemic event (e.g., stroke) that creates or embodies a risk of neurological damage in Central Nervous System (“CNS”) sites.

### BRIEF DESCRIPTION OF THE DRAWING

FIG. 1: Presents the canonical amino acid sequence of natural Tpo.

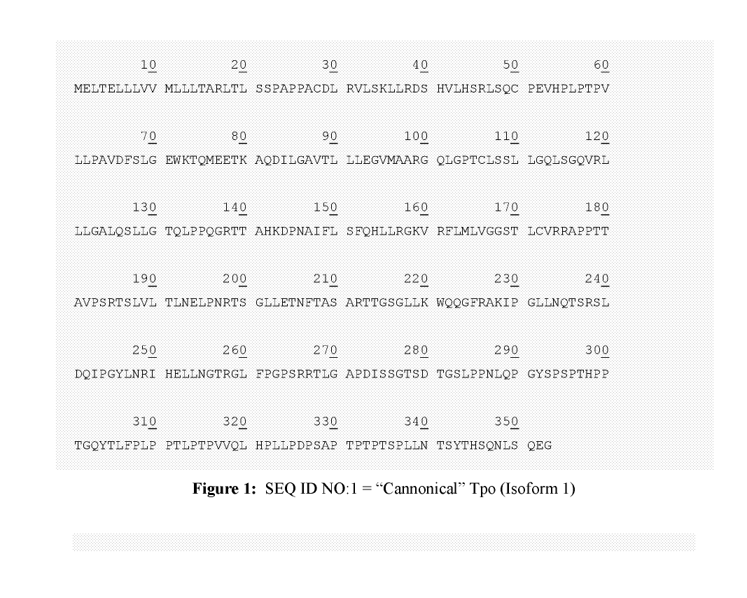

FIG. 2: Presents the amino acid sequence of “Isoform 2” of Tpo.

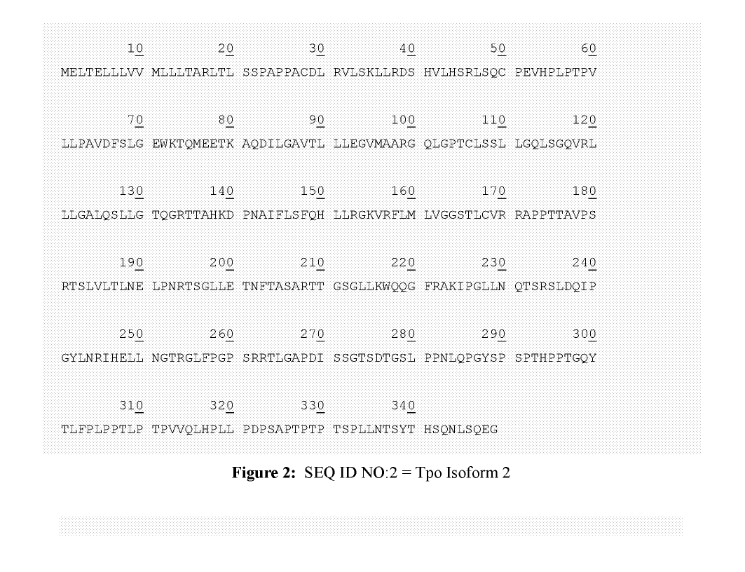

FIG. 3: Presents the amino acid sequence of a truncated Tpo.

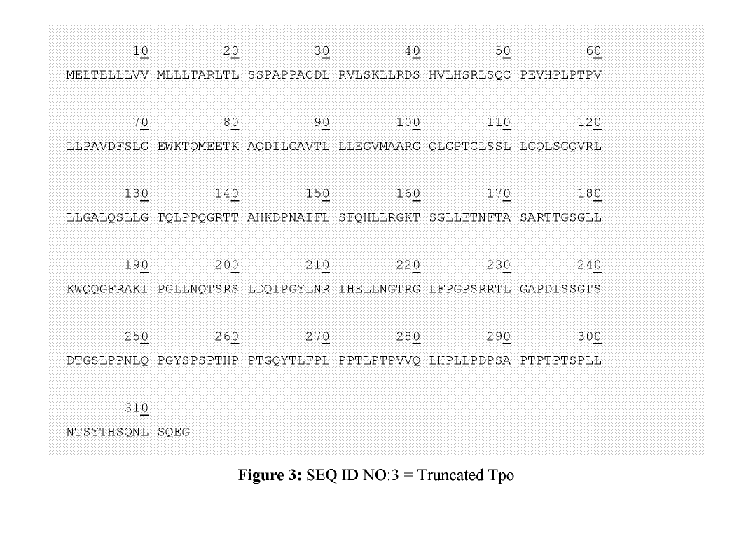

### DEFINITIONS

Associated with: The term “associated with”, in its most general sense, refers to any direct or indirect attachment between two (or more) entities. In some embodiments, the entities are directly associated with one another in that there is no intervening entity (e.g., linker). In some embodiments, entities are considered to be directly associated with one another if they are covalently bound to one another. In some embodiments, an association is or comprises one or more covalent bonds. In some embodiments, an association is or comprises one or more non-covalent interactions (e.g., involving one or more of hydrophobic forces, van der Waals forces, hydrogen bonds, magnetic interactions, etc). In some embodiments, associated entities are reversibly associated with one another in that the association can be disrupted under certain (typically predetermined) conditions. In some embodiments, the reversible associations are or comprise associations selected from the group consisting of electrostatic bonding, hydrogen bonding, van der Walls forces, ionic interaction, or donor/acceptor bonding. In some embodiments, reversible association can include a combination of interactions, such as a combination of hydrogen bonding and ionic bonding, etc. In some embodiments, entities are irreversibly associated with one another. In some embodiments, an association involves specific binding.

Characteristic sequence element: As used herein, the phrase a “characteristic sequence element” of a protein or polypeptide is one that contains a continuous stretch of amino acids, or a collection of continuous stretches of amino acids, that together are characteristic of a protein or polypeptide. Each such continuous stretch generally will contain at least two amino acids. Furthermore, those of ordinary skill in the art will appreciate that typically at least 5, at least 10, at least 15, at least 20 or more amino acids are required to be characteristic of a protein. In general, a characteristic sequence element is one that, in addition to the sequence identity specified above, shares at least one functional characteristic (e.g., biological activity, epitope, etc) with the relevant intact protein. In many embodiments, a characteristic sequence element is one that is present in all members of a family of polypeptides, and can be used to define such members.

Combination therapy: The term “combination therapy”, as used herein, refers to those situations in which two or more different pharmaceutical agents are administered in overlapping regimens so that the subject is simultaneously exposed to both agents.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regime comprises a plurality of doses and at least two different time periods separating individual doses.

Subject: As used herein, the term “subject” or “patient” refers to any organism upon which embodiments of the invention may be used or administered, e.g., for experimental, diagnostic, prophylactic, and/or therapeutic purposes. Typical subjects include animals (e.g., mammals such as mice, rats, rabbits, non-human primates, and humans; insects; worms; etc.).

Suffering from: An individual who is “suffering from” a disease, disorder, or condition (e.g., stroke) has been diagnosed with and/or exhibits one or more symptoms of the disease, disorder, or condition.

Therapeutic regimen: As used herein, the term “therapeutic regimen” refers to any protocol used to partially or completely alleviate, ameliorate, relieve, inhibit, prevent, delay onset of, reduce severity of and/or reduce incidence of one or more symptoms or features of a particular disease, disorder, and/or condition. In some embodiments, a therapeutic regimen may comprise a treatment or series of treatments whose administration correlates with achievement of a particular result across a relevant population. In some embodiments, a therapeutic regimen involves administration of one or more therapeutic agents, either simultaneously, sequentially or at different times, for the same or different amounts of time. Alternatively, or additionally, the treatment may include exposure to protocols such as radiation, chemotherapeutic agents or surgery. Alternatively or additionally, a “treatment regimen” may include genetic methods such as gene therapy, gene ablation or other methods known to reduce expression of a particular gene or translation of a gene-derived mRNA.

Therapeutic agent: As used herein, the phrase “therapeutic agent” refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” refers to an amount of a therapeutic agent whose administration, when viewed in a relevant population, correlates with or is reasonably expected to correlate with achievement of a particular therapeutic effect. The therapeutic effect may be objective (i.e., measurable by some test or marker) or subjective (i.e., subject gives an indication of or feels an effect). A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific fusion protein employed; the duration of the treatment; and like factors as is well known in the medical arts.

Treatment: As used herein, the term “treatment” refers to a therapeutic protocol that alleviates, delays onset of, reduces severity or incidence of, and/or yield prophylaxis of one or more symptoms or aspects of a disease, disorder, or condition. In some embodiments, treatment is administered before, during, and/or after the onset of symptoms. In some embodiments, treatment may be administered to a subject who does not exhibit signs of a disease, disorder, and/or condition. In some embodiments, treatment may be administered to a subject who exhibits only early signs of the disease, disorder, and/or condition, for example for the purpose of decreasing risk of developing pathology associated with the disease, disorder, and/or condition.

### DETAILED DESCRIPTION OF CERTAIN EMBODIMENTS

### Stroke

Two of the most common types of strokes are ischemic strokes and hemorrhagic strokes. In ischemic strokes, a lack of oxygen flow to the brain can result in apoptosis and necrosis of brain tissue leading to infarction. Similar to cardiovascular ischemia, brain ischemia can be caused by various factors such as blood clots, thrombosis, embolism, blockage by atherosclerotic plaques, or other obstructions in the vasculature. Hypercholesterolemia, hypertension, diabetes, and obesity, among other things, have been identified as risk factors for ischemic strokes. Ischemic strokes are a leading cause of death of human beings worldwide.

Hemorrhagic strokes, which account for between about 10 and 20 percent of all strokes, are typically caused by a ruptured blood vessel in the brain. The rupture causes bleeding into the brain, where the accumulating blood can damage surrounding neural tissues.

The stroke episode, regardless of its cause, results in neural cell death, especially at the location of the obstruction or hemorrhage. In addition, biochemical reactions that occur subsequent to the stroke episode in the vasculature may lead to edema, hemorrhagic transformation, and a further compromise in neurological tissue. The neurological damage and neuron cell death that result from a stroke can be physically and mentally debilitating to an individual. Among other things, a stroke can result in problems with emotional control, awareness, sensory perception, speech, hearing, vision, cognition, movement and mobility, and can cause paralysis.

### Thrombopoietin Receptor Ligands

The present invention encompasses the recognition that thrombopoietin receptor ligands are useful in the treatment or prevention of damage due to an ischemic event (e.g., stroke) that creates or embodies a risk of neurological damage in CNS sites and/or to stroke. In some embodiments, the present invention encompasses the recognition that thrombopoietin receptor ligands can protect CNS tissues (e.g., the brain) against injury from an ischemic event (e.g., stroke) that creates or embodies a risk of neurological damage in CNS sites and/or from stroke. In some embodiments, provided therapy protects against one or more of the above-mentioned problems with emotional control, awareness, sensory perception, speech, hearing, vision, cognition, movement and mobility, and can cause paralysis, and/or against tissue damage resulting from the ischemic event and/or stroke.

Thrombopoietin (also known as Tpo, c-mpl ligand, megakaryocyte growth and differentiation factor, thrombocytopoiesis stimulating factor) is a glycopeptide hormone of approximately 70,000 molecular weight. Tpo is the ligand for the Mpl cytokine receptor, and is the primary physiologic regulator of megakaryocyte and platelet development. Tpo is naturally produced in the liver, and also in the kidneys and/or bone marrow. Tpo circulates in blood plasma with a normal concentration range of 50-250 pg/ml.

Tpo is synthesized as a 353 amino acid precursor protein with a molecular weight of 36 kDa. Following the removal of a 21 amino acid signal peptide, the remaining 332 amino acids undergo glycosylation to produce a 95 kDa glycoprotein. Mature Tpo can be divided into two domains: the amino-terminal half with homology to erythropoietin (Epo) and the carboxy-terminal half rich in serine, threonine and proline residues and containing seven potential N-linked glycosylation sites. The carboxy terminus domain of TPO has been shown to regulate the specific activity and circulating half-life of TPO. The carboxy-terminal may also have a role in promoting the efficient biosynthesis and secretion of TPO. The glycoprotein is then released into the circulation; it is not known to be stored in the liver or kidney.

The Mpl receptor is expressed on platelets, megakaryocytes, and all stages of megakaryocyte progenitors, including CD34+ repopulating stem cells. The present inventor has also demonstrated that Tpo receptor is present in the heart. Moreover, the present inventor has demonstrated that Tpo receptor ligands can provide protection against damage from ischemic injury, and particularly can provide protection again cardiac injury from ischemic events (i.e., can protect heart tissue from damage caused by myocardial ischemia; see U.S. Pat. No. 7,879,318). The inventor has specifically shown that Tpo protects the heart against various deleterious effects of ischemia/reperfusion.

The present invention represents an extension of these insights, and encompasses the inventor's further insight that CNS (e.g., brain) tissues may express the Tpo receptor, such that they may be able to respond to appropriate administration of a Tpo receptor ligand. According to the present invention, such administration can reduce the frequency and/or severity of, and/or can delay onset of damage to CNS tissues resulting from an ischemic event (e.g., stroke) that creates or embodies a risk of neurological damage in CNS sites, as described herein.

The present invention further encompasses the inventor's recognition that challenges may be encountered in achieving prompt or immediate Neuroprotective effects in central nervous system sites. The present invention specifically encompasses the recognition that certain Tpo receptor ligands show poor blood brain barrier penetration. The invention provides strategies for achieving neuroprotection (e.g., prompt neuroprotective effects) in CNS target sites. Among other things, the present invention provides dosing compositions and regimens that deliver amounts of Tpo receptor ligands sufficient to provide neuroprotective effects within a relevant time window of an ischemic event (e.g., an event that creates or embodies a risk of neurological damage in CNS sites).

As will be clear to those of ordinary skill in the art, any Tpo receptor ligand, appropriately administered, is useful as described herein. In some embodiments, a Tpo receptor ligand as described herein binds to the Tpo receptor. In some embodiments, binding of a Tpo receptor ligand as described herein triggers one or more biological events comparably triggered by binding of Tpo to the Tpo receptor. In some embodiments, a Tpo receptor ligand as described herein is an agent that binds to the Tpo receptor and triggers one or more biological events that is comparably triggered by binding of Tpo to the receptor, so that the biological event occurs at a level at least 50%, 55%, 60%, 65%, 70%, 75%, 80%, 85%, 90%, 95%, 96%, 97%, 98%, 99% 100%, or more of that at which it occurs when Tpo binds to the receptor under comparable conditions.

In some embodiments, a Tpo receptor ligand as described herein is an agent that competes with Tpo for binding to its receptor. In some embodiments, Tpo binding to its receptor is reduced at least about 10%, 15%, 20%, 25%, 30%, 35%, 40%, 45%, 50%, 55%, 60%, 65%, 70%, 75%, 80%, 85%, 90%, 95%, 96%, 97%, 98%, 99% or more in the presence of a Tpo receptor ligand, as compared with in its absence.

Tpo itself can be obtained from any of a variety of sources (e.g., Genentech of South San Francisco, Pfizer of New York, N.Y.), and/or can be prepared according to any of a variety of known processes (see, for example, U.S. Pat. No. 5,830,647 by Easton et al., U.S. Pat. No. 5,879,673 by Thomas et al.). Tpo has been purified and cloned from several species including mouse, rat, and dog (see References below). Tpo proteins from the various species are highly conserved, exhibiting from 69-75% sequence identity at the amino acid sequence level.

Various Tpo derivatives or analogs are known in the art and are useful in accordance with the present invention to the extent that they are Tpo receptor ligands. In some embodiments, a Tpo derivative or analog is a polypeptide that shows a specified overall degree of sequence identity with Tpo and/or shares at least one characteristic sequence element with Tpo (e.g., with SEQ ID NO:1). In some embodiments, a Tpo derivative or analog shows at least 45%, 50%, 55%, 60%, 65%, 70%, 75%, 80%, 85%, 90%, 95%, 96%, 97%, 98%, 99% or more overall sequence identity with SEQ ID NO:1, or with a least 100 consecutive residues thereof. In some embodiments, a Tpo derivative or analog shows at least about 70% overall sequence identity with SEQ ID NO:1. In some embodiments, a Tpo derivative or analog useful in accordance with the present invention shares at least one characteristic sequence element with Tpo.

Certain Tpo receptor ligands that may be useful in accordance with the present invention may include one or more Tpo-related agents that are modified as compared with Tpo, for example in order to improve thrombopoietic activity as compared with a reference Tpo (e.g., of SEQ ID NO:1). Certain such agents include Tpo derivated with amino acids at the carboxy terminus.

Alternatively or additionally, Tpo receptor ligands for use in accordance with the present invention include Tpo isoforms with various numbers of sialic acid residues per molecule.

Alternatively or additionally, Tpo receptor ligands for use in accordance with the present invention may include peptides which bind to the Tpo receptor, whether or not such polypeptides show amino acid sequence identity with Tpo (e.g., SEQ ID NO:1) itself. Certain such Tpo receptor ligand polypeptides are described in one or more of U.S. Pat. Nos. 5,869,451, 5,932,546, 6,083,913, 6,121,238, 5,869,451, 6,251,864 6,506,362 and 6,465,430 and U.S. Pub. No. U.S. 2003/0158116; such polypeptides may be exemplified by compound 497115 from Glaxo Smith Kline.

Alternatively or additionally, Tpo receptor ligands for use in accordance with the present invention may include one or more small-molecule Tpo mimetics, for example as described in one or more of U.S. Pub. Nos. U.S. 2003/0195231, U.S. 2003/0162724, U.S. 2004/0063764, U.S. 2004/0082626 and/or in one or more of U.S. Pat. Nos. 7,790,704, 7,795,293, 7,160,870, 7,332,481, 7,452,874, 7,473,686.

Alternatively or additionally, Tpo receptor ligands for use in accordance with the present invention may include polypeptides (e.g., Tpo polypeptides including for example Tpo derivatives or analogs as described herein) modified with polyethylene glycol, and/or with glycosylation, for example as described by Elliot, et al. (Nature Biotechnology 21:414-421, 2003), AMG531 (Amgen, Thousand Oaks, Calif.).

Alternatively or additionally, Tpo receptor ligands for use in accordance with the present invention may include a Tpo agonist antibody described by Alexion Pharmaceuticals (Cheshire, Conn.) and/or Xoma (Berkeley, Calif.) and/or described in U.S. Pub. No. U.S. 2004/0136980.

Alternatively or additionally, Tpo receptor ligands for use in accordance with the present invention may include polypeptides (e.g., Tpo polypeptides including for example Tpo derivatives or analogs as described herein) modified by carbamylation, succinylation, acetylation, biotinylation, iodination, carboxyinethyllysylatkl, and/or the like.

Alternatively or additionally, Tpo receptor ligands for use in accordance with the present invention may be or comprised those described and/or claimed in one or more of U.S. Pat. Nos. 6,887,890; 5,989,538; 5,756,083; 5,498,599; and 6,866,998.

Alternatively or additionally, Tpo receptor ligands for use in accordance with the present invention may be or include one or more of those described in:
1. Wang, B, et al., Clin. Pharmacol. Ther. 73:628-638, 2004.
2. Orita, T., et al., Blood 105:562-566, 2005.
3. Inagaki, K., et al., Blood 104:58-64, 2004.
4. de Serres, M., et al., Stem Cells 17:203-209, 1999.
5. de Serres, M., et al., Stem Cells 17:316-326, 1999.
6. Cwirla, S. E., et al., Science 276:1696-1699, 1997.
7. Case, B. C., et al., Stem Cells 18:360-365, 2000.
8. Erickson-Miller, C. L., et al., Exp. Hematol. 33:85-93, 2005.

### Dosing and Administration

In accordance with the present invention, a Tpo receptor ligand is administered to a subject who is suffering or has suffered an ischemic event (e.g., stroke) that creates or embodies a risk of neurological damage in CNS sites, so that the Tpo receptor ligand binds to Tpo receptors in CNS tissue (e.g., brain tissue) and protects that tissue from one or more aspects or features of damage from the ischemic event. In some embodiments, a Tpo receptor ligand is administered to a subject who is experiencing or has recently experienced one or more symptoms of an ischemic event (e.g., stroke) that creates or embodies a risk of neurological damage in CNS sites. In some embodiments, a Tpo receptor ligands is administered to a subject who is not in need of a platelet increase. In some embodiments, a Tpo receptor ligand is administered to a subject whose platelet count is within the normal range (i.e., within about 150,000 to about 400,000 per microliter). In some embodiments, at least one dose of a Tpo receptor ligand is administered, and dosing is stopped when a neuroprotective benefit has been achieved or is expected to be achieved (e.g., because a number and type of doses has been administered that together correlate across a population with achievement of a particular CNS benefit).

In some embodiments, a Tpo receptor ligand is administered locally to a site in the CNS (e.g., by intracavitary, intrathecal, or other local mode of delivery)

In some embodiments, a Tpo receptor ligand is administered systemically (e.g., parenterally, orally, mucosally, etc) such that (e.g., as part of a formulation that ensures) the Tpo receptor ligand crosses the blood-brain barrier.

In some embodiments, a Tpo receptor ligand is administered in accordance with the present invention by a regimen that does not increase platelet production more than about 1% after any single dose in the regimen. Those of ordinary skill in the art will appreciate that absolute dose amounts of Tpo receptor ligand as described herein are expected to be different for different Tpo receptor ligand entities. Those of ordinary skill in the art will further appreciate that a useful dose, or dosing regimen, for any particular Tpo receptor ligand as described herein may usefully be defined with reference to a “comparable” dose of a reference Tpo receptor ligand. In some embodiments, the reference Tpo is natural Tpo. In some embodiments, the reference Tpo is a polypeptide whose amino acid portion is has or includes a sequence as set forth in SEQ ID NO:1. Thus, for example, in some embodiments, provided compositions deliver an amount of a Tpo receptor ligand that corresponds to a particular recited amount of Tpo of SEQ ID NO:1. Those of ordinary skill in the art will appreciate that a dose of one Tpo receptor ligand “corresponds to” a dose of a different Tpo receptor ligand if and when administration of each achieves a comparable (e.g., within a reasonable variation as determined by the assay at hand) degree or extent of modulation of a biological process or event.

In some embodiments, the present invention provides unit dosage forms comprising a Tpo receptor ligand and a pharmaceutically acceptable carrier. In some such embodiments, the unit dosage form contains an amount of Tpo receptor ligand sufficient to achieve binding to its receptor on the surface of cells in CNS (e.g., brain) tissue. Without wishing to be bound by any particular theory, we propose that such binding results in activation of Janus kinase, which in turn activates cell survival mechanisms that protect against injury.

In some embodiments, a provided unit dosage form is formulated and contains an amount of Tpo receptor ligand sufficient to achieve blood levels in the CNS (e.g., in the brain) in a range of 0.01-10.0 ng Tpo receptor ligand/ml or 0.05-0.5 ng Tpo receptor ligand/ml or 0.5-5.0 ng Tpo receptor ligand/ml, or about 1.0 ng Tpo receptor ligand/ml. In some embodiments, a provided unit dosage form contains an amount of Tpo receptor ligand sufficient to achieve blood levels of the Tpo receptor ligand in the CNS (e.g., in the brain) that correspond to levels of Tpo (e.g., SEQ ID NO:1) in a range of 0.01-10.0 ng Tpo/ml or 0.05-0.5 ng Tpo/ml or 0.5-5.0 ng Tpo/ml, or about 1.0 ng Tpo/ml.

In some embodiments, a provided unit dosage form contains an amount of Tpo receptor ligand sufficient to achieve systemic blood levels of Tpo receptor ligand that are materially higher than the CNS blood levels (e.g., due to poor blood-brain barrier penetrance and/or other reasons). In some embodiments, a provided unit dosage form contains an amount of TPO receptor ligand sufficient to achieve systemic blood levels of Tpo receptor ligand that are at least 1, 2, 3, 4, 5, 6, 7, 8, 9, 0, 20, 25, 30, 35, 40, 45, 50, 55, 60, 65, 70, 75, 80, 85, 90, 95, 100, 200, 300, 400, 500 750, 1000 fold or more higher than the CNS blood level (e.g., than a CNS blood level noted above).

In some embodiments, desired levels of Tpo receptor ligand are achieved substantially immediately after administration. For example, in some embodiments, such levels are achieved within about 35 minutes of administration. In some embodiments, such levels are achieved within about 1-20 minutes, 1-15 minutes, 1-10 minutes, 1-5 minutes, or even fewer.

In some embodiments, provided unit dosage forms contain an amount of Tpo receptor ligand that corresponds to a dose (e.g., a systemic dose) of Tpo (e.g., of SEQ ID NO:1 within the range of 1-1000 mcg/kg. In some embodiments, provided unit dosage forms contain an amount of Tpo receptor ligand that corresponds to a dose of Tpo (e.g., of SEQ ID NO:1) within a range that has a lower boundary of 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 mcg/kg or more, and has an upper boundary of 1000, 950, 900, 850, 800, 750, 700, 650, 600, 550, 500, 450, 400, 350, 300, 250, 200, 150, or 100 mcg/kg.

In some embodiments, provided unit dosage forms contain an amount of Tpo receptor ligand that corresponds to a dose (e.g., a local dose) of Tpo (e.g., of SEQ ID NO:1) within the range of 0.6-60 mcg/kg. In some embodiments, provided unit dosage forms contain an amount of Tpo receptor ligand that corresponds to a dose of Tpo (e.g., of SEQ ID NO:1) within a range that has a lower boundary of 0.1, 0.2, 0.3, 0.4, 0.5, 0.6, 0.7, 0.8, 0.9, 1.0. 1.5, 2.0, 2.5, 3.0, 3.5, 4.0, 4.5, or 5.0, and has an upper boundary of 100, 95, 90, 85, 80, 75, 70, 65, 60, 55, 50, 45, 40, 35, 30, 25, 20, 15, 10, or 5 mcg/kg.

In some embodiments, a provided unit dosage form contains an amount of Tpo receptor ligand that corresponds to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10000, 11000, 12000, 13000, 14000, 15000, 16000, 17000, 180900, 19000, 20000, 25000, 30000, 35000, 40000, 45000, 50000, 55000, 60000, 65000, 70000, 75000, 80000 85000, 90000, 95000, 100000, or more mcg Tpo (e.g., SEQ ID NO:1).

In some embodiments, the present invention provides methods that involve administration of a single dose of Tpo receptor ligand. In some embodiments, the present invention provides methods that involve administration of a plurality of doses of Tpo receptor ligand, spaced apart from one another in time. In some embodiments, the present invention provides methods that involve administration of a plurality of identical doses of Tpo receptor ligand. In some embodiments, the present invention provides methods that involve administration of a plurality of doses that are not all identical (i.e., that may differ from one another in amount, route, timing, and/or any of a number of other variables as are known to those skilled in the art). In some embodiments, the present invention provides methods that involve administration of a first dose of a first amount, followed by a plurality of doses that are identical to or different from one another, but are different from the first dose. In some such embodiments, the first dose is larger than one or more subsequent doses. In some such embodiments, successive doses increase in amount.

In some embodiments, the present invention provides methods that involve administration of Tpo receptor ligand such that at least one dose is administered within a predetermined period of time (i.e., time window) after onset of an ischemic event (e.g., stroke) that creates or embodies a risk of neurological damage in CNS sites (e.g., of one or more symptoms of a stroke). In some embodiments, all doses of Tpo receptor ligand are administered within the predetermined period of time after onset of a relevant ischemic event. In some embodiments, the predetermined period of time is about 10, 15, 20, 25, 30, 25, 40, 45, 50, 55, 60, 90, 120 minutes or more. In some embodiments the predetermined period of time is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 42, 42, 43, 44, 45, 46, 47, 48, 49 hours, or more.

In some embodiments, not more than one dose of Tpo receptor ligand is administered in a day. In some embodiments, all doses within a regimen are the same amount. In some embodiments, different doses are or include different amounts. In some embodiments, different doses are spaced apart from one another by the same period of time. In some embodiments, different periods of time separate different doses within a regimen.

Those of ordinary skill in the art will readily appreciate that, for any given Tpo receptor ligand, the appropriate amount to be included in a unit dosage form of in accordance with the present invention may well vary in light of, for example, route of delivery (e.g., intracavitary vs oral vs parenteral, etc).

Those of ordinary skill in the art will further appreciate that dosing regimens as provided in accordance with the present invention differ materially from current therapeutic regimens for Tpo receptor ligands for use in other indications, and specifically from those for use in increasing platelet production.

For example, one Tpo receptor ligand known in the art is romiplostim (Nplate™; provided by Amgen), which is an Fc fusion peptide (specifically a C-terminal dimer) that is approved for use to improve platelet count and is provided in 250 mcg and 500 mcg single use vials for subcutaneous administration to subjects suffer from low platelet count (and do not have a platelet count above 400×10⁹).

Another Tpo receptor ligand known in the art is eltrombopag (Promacta™; provided by GlaxoSmithKline), which is a Tpo mimetic for treatment of thrombocytopenia in patients with chronic immune thrombocytopenia purpura who have had an insufficient response to corticosteroids, immunoglobulins, or splenectomy. Eltrombopag is provided in 25 mg, 50 mg, and 75 mg tablets, and is administered via an initial dose (typically 50 mg once daily), that is later adjusted to achieve platelet count about 50×10⁹.

Another Tpo receptor ligand known in the art is E-5501 (provided by Eisai, Inc), which is administered for thrombocytopenia related to chronic liver disease or for autoimmune thrombocytopenic purpura. For thrombocytopenia related to chronic liver disease, E-5501 is administered orally according to a regimen comprising a first dose of 80 mg followed by either 10 mg a day for 6 days or 20 mg a day for 3 days. For autoimmune thrombocytopenic purpura, E-5501 is administered orally according to a regimen comprising a first dose of 20 mg followed by subsequent doses that can be titrated up to a maximum of 40 mg or down to a minimum of 5 mg. The goal is to maintain the plateley count at levels between 50×10⁹/L and 150×10⁹/L. Dosing should not be administered to subjects whose platelet could is greater than or equal to 35×10⁹/L prior to therapy.

### Pharmaceutical Compositions

In some embodiments, Tpo receptor ligand the present invention provides Tpo receptor ligands in pharmaceutical compositions formulated to achieve delivery of the Tpo receptor ligand in the CNS (e.g., brain). In some embodiments, the pharmaceutical composition is formulated to deliver the Tpo receptor ligand across the blood brain barrier. As is known in the art, different Tpo receptor ligands may have different levels of ability to cross the blood-brain barrier. For example, eltrombopag and romiplostim each cross the blood brain barrier more effectively than does Tpo itself (see, for example, Blood 85:981; J interferon Cyto Res).

The present invention provides pharmaceutical compositions of a Tpo receptor ligand and at least one pharmaceutically acceptable carrier or excipient, optionally formulated to achieve delivery across the blood-brain barrier.

In the present invention, Tpo receptor ligand is formulated in a pharmaceutical composition by combining the Tpo receptor ligand with a pharmaceutically acceptable carrier in a therapeutic amount effective to reduce myocardial ischemia in a patient to decrease damage to the heart.

In some embodiments, a pharmaceutically acceptable carrier is selected from the group consisting of sterile distilled water, saline, Ringer's solution, dextrose solution, Hank's solution, or the like, and physiologically acceptable to the patient.

In some embodiments (e.g., for parenteral administration), the Tpo receptor ligand can, for example, be incorporated into a solution or suspension, preferably a buffered solution or suspension.

A intranasal formulation can be prepared, for example, as a solution or suspension for delivery in the form of drops or spray using, for example, a nebulizer or atomizer for inhalation by the patient. A parenteral or intranasal preparation can be aseptically enclosed in ampoules, vials, disposable syringes, and other suitable containers.

In a transdermal delivery system, a provided Tpo receptor ligand can be formulated as a topical composition in a liquid or semi-liquid form such as a lotion, cream, ointment, gel, paste, solution or suspension. In some embodiments, transdermal delivery of a Tpo receptor ligand by skin penetration can be enhanced by use of occlusive techniques (e.g., wrap or impermeable plastic film) that hydrate the skin and increase skin temperature, or by the use of a suitable penetrating agent (e.g., water, polyols such as glycerin and propylene glycol).

A suppository dosage form can be prepared, for example, by combining a Tpo receptor ligand with a carrier comprising a cocoa butter base, or a water-soluble or dispersible base such as polyethylene glycols and glycerides, that is solid at room temperature (about 20° C.) and melts at body temperature. Suppositories are typically individually foil wrapped, or hermetically sealed in a molded plastic container.

### Combination Therapy

In some embodiments, Tpo receptor ligand therapy as described herein is administered in combination with one or more other agents, for example that may alleviate one or more symptoms or effects of an ischemic event (e.g., stroke) that creates or embodies a risk of neurological damage in CNS sites.

To give but a few examples, in some embodiments, Tpo receptor therapy as described herein is administered in combination with one or more pain relievers, anti-inflammatories, immunomodulaors, blood thinners, thrombolytics, etc.

### EXEMPLIFICATION

### Example 1

### Protective Efficacy and Mechanism of Tpo Intervention in Focal Stroke

The present Example describes studies that confirm, as described herein, that Tpo receptor ligands are useful in the treatment or prevention of one or more negative effects of stroke. The Example specifically confirms that Tpo receptor ligands can reduce incidence or severity of and/or delay onset of one or more aspects or features of CNS (e.g., brain) tissue damage associated with stroke.

Male Sprague Dawley rats, 10 weeks of age (n=6/group) undergo 2-3 hours of left middle cerebral artery occlusion followed by 20-48 hours of reperfusion. Vehicle or thrombopoietin (0.03 to 1.00 μg/kg) is administered intravenously immediately after reperfusion. Brain infarct and swelling, neurologic deficits, matrix metalloproteinase-9 (MMP-9), tissue inhibitor of metalloproteinase-1 (TIMP-1), thrombopoietin and c-Mpl (thrombopoietin receptor) mRNA and protein, MMP-9 enzyme activity and protein expression, and the integrity of the blood-brain barrier are measured. Reperfusion of the left middle cerebral artery occlusion is expected to produce a large infarct and swelling after stroke. The expected outcome of the studies is that thrombopoietin significantly reduces these indices of injury to the brain in a dose-dependent manner.

Thrombopoietin is administered intravenously over the range 0.001-10 μg/kg. The most effective thrombopoietin dose is in the range 0.05-1.0 μg/kg, when administrated immediately or 2 hours after reperfusion, which would significantly reduce infarct size and swelling and ameliorate neurologic deficits after stroke when compared with untreated controls. Stroke-induced increases in cortical MMP-9 mRNA, enzyme activity and protein expression, TIMP-1 mRNA, and Evans blue extravasation would be reduced by administration of thrombopoietin. Thrombopoietin would not be expected to alter cortical thrombopoietin or c-Mpl (thrombopoietin receptor) mRNA and protein expression, blood pressure, heart rate, blood hematocrit, or platelet count.

Results of this study would be expected to provide the first confirmation of thrombopoietin's efficacy in reducing ischemic brain injury and improving functional outcome. Without wishing to be bound by any particular theory, Applicant proposes that at least some beneficial effects of Tpo can be attributed to its reducing stroke-induced increases in one or both of 1) MMP-9 level and/or activity, and 2) blood-brain barrier dysfunction.

### REFERENCES

[1] “Stimulation of megakaryocytopoiesis and thrombopoiesis by the c-Mpl ligand.”
   de Sauvage F. J., Hass P. E., Spencer S. D., Malloy B. E., Gurney A. L., Spencer S. A., Darbonne W. C., Henzel W. J., Wong S. C., Kuang W.-J., Oles K. J., Hultgren B., Solberg L. A. Jr., Goeddel D. V., Eaton D. L.
   Nature 369:533-538(1994) [PubMed: 8202154] [Abstract]
   Cited for: NUCLEOTIDE SEQUENCE [MRNA] (ISOFORM 1).
   Tissue: Fetal liver.
[2] “Identification and cloning of a megakaryocyte growth and development factor that is a ligand for the cytokine receptor Mpl.”
   Bartley T. D., Bogenberger J., Hunt P., Li Y.-S., Lu H. S., Martin F., Chang M.-S., Samal B. B., Nichol J. L., Swift S., Johnson M. J., Hsu R.-Y., Parker V. P., Suggs S., Skrine J. D., Merewether L. A., Clogson C., Hsu E. [symbol] Bosselman R. A.
   Cell 77:1117-1124(1994) [PubMed: 8020099] [Abstract]
   Cited for: NUCLEOTIDE SEQUENCE [MRNA] (ISOFORM 1).
   Tissue: Fetal liver.
[3] “Human thrombopoietin: gene structure, cDNA sequence, expression, and chromosomal localization.”
   Foster D. C., Sprecher C. A., Grant F. J., Kramer J. M., Kuijper J. L., Holly R. D., Whitmore T. E., Heipel M. D., Bell L. A. N., Ching A. F., McGrane V., Hart C., O'Hara P. J., Lok S.
   Proc. Natl. Acad. Sci. U.S.A. 91:13023-13027(1994) [PubMed: 7809166] [Abstract]
   Cited for: NUCLEOTIDE SEQUENCE [GENOMIC DNA/MRNA] (ISOFORM 1).
[4] “Molecular cloning and chromosomal localization of the human thrombopoietin gene.”
   Sohma Y., Akahori H., Seki N., Hori T.-A., Ogami K., Kawamura K., Miyazaki H.
   FEBS Lett. 353:57-61(1994) [PubMed: 7926023] [Abstract]
   Cited for: NUCLEOTIDE SEQUENCE [GENOMIC DNA] (ISOFORM 1).
[5] “Genomic structure, chromosomal localization, and conserved alternative splice forms of thrombopoietin.”
   Gurney A. L., Kuang W.-J., Xie M.-H., Malloy B. E., Eaton D. L., de Sauvage F. J.
   Blood 85:981-988(1995) [PubMed: 7849319] [Abstract]
   Cited for: NUCLEOTIDE SEQUENCE [GENOMIC DNA] (ISOFORMS 1 AND 2).
[6] “Purification and characterization of thrombopoietin.”
   Kato T., Ogami K., Shimada Y., Iwamatsu A., Sohma Y., Akahori H., Hone K., Kokubo A., Kudo Y., Maeda E., Kobayashi K., Ohashi H., Ozawa T., Inoue H., Kawamura K., Miyazaki H.
   J. Biochem. 118:229-236(1995) [PubMed: 8537317] [Abstract]
   Cited for: NUCLEOTIDE SEQUENCE [MRNA] (ISOFORM 1).
   Tissue: Liver.
[7] “Cloning and characterization of the human megakaryocyte growth and development factor (MGDF) gene.”
   Chang M., McNinch J., Basu R., Shutter J., Hsu R., Perkins C., Mar V., Suggs S., Welcher A., Li L., Lu H., Bartley T., Hunt P., Martin F., Samal B., Bogenberger J.
   J. Biol. Chem. 270:511-514(1995) [PubMed: 7822271] [Abstract]
   Cited for: NUCLEOTIDE SEQUENCE [GENOMIC DNA] (ISOFORM 1).
   Tissue: Placenta.
[8] “Cloning and sequencing of human thrombopoietin.”
   Im S. H., Lee W. S., Chung K. H.
   Submitted (May-1996) to the EMBL/GenBank/DDBJ databases
   Cited for: NUCLEOTIDE SEQUENCE [GENOMIC DNA] (ISOFORMS 1 AND 3).
[9] “The status, quality, and expansion of the NIH full-length cDNA project: the Mammalian Gene Collection (MGC).”
   The MGC Project Team
   Genome Res. 14:2121-2127(2004) [PubMed: 15489334] [Abstract]
   Cited for: NUCLEOTIDE SEQUENCE [LARGE SCALE MRNA] (ISOFORMS 1 AND 2).
   Tissue: Brain and Testis.
[10] “An activating splice donor mutation in the thrombopoietin gene causes hereditary thrombocythaemia.”
   Wiestner A., Schlemper R. J., van der Maas A. P. C., Skoda R. C.
   Nat. Genet. 18:49-52(1998) [PubMed: 9425899] [Abstract]
   Cited for: DISEASE.
[11] “Peptide, disulfide, and glycosylation mapping of recombinant human thrombopoietin from serl to Arg246.”
   Hoffman R. C., Andersen H., Walker K., Krakover J. D., Patel S., Stamm M. R., Osborn S. G.
   Biochemistry 35:14849-14861(1996) [PubMed: 8942648] [Abstract]
   Cited for: DISULFIDE BONDS, GLYCOSYLATION AT SER-22; THR-58; THR-131; THR-179; THR-180; SER-184; ASN-197; ASN-206; THR-213; ASN-234; ASN-255 AND SER-265.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method comprising a steps of:
systemically administering to a subject who is suffering from or has recently suffered a hemorrhagic stroke, a composition comprising a thrombopoietin receptor ligand, wherein the composition is formulated and contains an amount of thrombopoietin receptor ligand sufficient to achieve delivery of the thrombopoietin receptor ligand to the central nervous system (“CNS”).

2. The method of claim wherein the step of administering comprises administering at least one dose of the composition within a time window that is not more than 35 minutes after onset of symptoms of the hemorrhagic stroke.

3. The method of claim 1, wherein the step of administering comprises administering only one dose.

4. The method of claim 1, wherein the step of administering comprises administering not more than one dose per day.

5. The method of claim 1, wherein the step of administering comprises administering a plurality of doses.

6. (canceled)

7. The method of claim 2, wherein the step of administering comprises administering only one dose.

8. The method of claim 2, wherein the step of administering comprises administering not more than one dose per day.

9. The method of claim 2, wherein the step of administering comprises administering a plurality of doses.

10. The method of claim 1, wherein the thrombopoietin receptor ligand is a recombinant human thrombopoietin (“rhTPO”).

11. The method of claim 10, wherein the thrombopoietin receptor ligand is truncated recombinant human thrombopoietin (“rhTPO”).

12. The method of claim 1, wherein the thrombopoietin receptor ligand is a Tpo polypeptide modified by carbamylation, succinylation, acetylation, biotinylation, iodination, glycosylation, pegylation, and/or carboxyinethyllysylation.

13. The method of claim 1, wherein the thrombopoietin receptor ligand is a Tpo mimetic.

14. The method of claim 1, wherein the thrombopoietin receptor ligand is a Tpo agonist antibody.

15. A method comprising a step of:
administering at least one dose of composition comprising eltrombopag to a subject who is suffering from or has recently suffered a hemorrhagic stroke.

16. The method of claim 15, wherein the administering is performed within a time period that is not more than 35 minutes after the hemorrhagic stroke.

17. The method of claim 15, wherein the eltrombopag is administered orally.

18. The method of claim 17, wherein the eltrombopag is administered at a dose of no more than 50 mg.

19. The method of claim 15, wherein the eltrombopag is administered parenterally.

20. The method of claim 19, wherein the eltrombopag is administered intravenously.

21. A method comprising a step of:
administering to a subject who is suffering from or has recently suffered a hemorrhagic stroke a composition comprising a thrombopoietin receptor ligand selected from the group consisting of eltrombopag and romiplostim, the composition being formulated and administered to achieve delivery of the Tpo receptor ligand in the CNS.


SEQUENCE LISTING (3 sequences)

SEQ ID NO: 1 - protein, 353 aa, Homo sapiens
MELTELLLVVMLLLTARLTLSSPAPPACDLRVLSKLLRDSHVLHSRLSQCPEVHPLPTPVLLPAVDFSLGEWKTQMEETKAQDILGAVTLLLEGVMAARGQLGPTCLSSLLGQLSGQVRLLLGALQSLLGTQLPPQGRTTAHKDPNAIFLSFQHLLRGKVRFLMLVGGSTLCVRRAPPTTAVPSRTSLVLTLNELPNRTSGLLETNFTASARTTGSGLLKWQQGFRAKIPGLLNQTSRSLDQIPGYLNRIHELLNGTRGLFPGPSRRTLGAPDISSGTSDTGSLPPNLQPGYSPSPTHPPTGQYTLFPLPPTLPTPVVQLHPLLPDPSAPTPTPTSPLLNTSYTHSQNLSQEG

SEQ ID NO: 2 - protein, 349 aa, Homo sapiens
MELTELLLVVMLLLTARLTLSSPAPPACDLRVLSKLLRDSHVLHSRLSQCPEVHPLPTPVLLPAVDFSLGEWKTQMEETKAQDILGAVTLLLEGVMAARGQLGPTCLSSLLGQLSGQVRLLLGALQSLLGTQGRTTAHKDPNAIFLSFQHLLRGKVRFLMLVGGSTLCVRRAPPTTAVPSRTSLVLTLNELPNRTSGLLETNFTASARTTGSGLLKWQQGFRAKIPGLLNQTSRSLDQIPGYLNRIHELLNGTRGLFPGPSRRTLGAPDISSGTSDTGSLPPNLQPGYSPSPTHPPTGQYTLFPLPPTLPTPVVQLHPLLPDPSAPTPTPTSPLLNTSYTHSQNLSQEG

SEQ ID NO: 3 - protein, 314 aa, Artificial Sequence
  Description of Artificial Sequence Synthetic polypeptide
MELTELLLVVMLLLTARLTLSSPAPPACDLRVLSKLLRDSHVLHSRLSQCPEVHPLPTPVLLPAVDFSLGEWKTQMEETKAQDILGAVTLLLEGVMAARGQLGPTCLSSLLGQLSGQVRLLLGALQSLLGTQLPPQGRTTAHKDPNAIFLSFQHLLRGKTSGLLETNFTASARTTGSGLLKWQQGFRAKIPGLLNQTSRSLDQIPGYLNRIHELLNGTRGLFPGPSRRTLGAPDISSGTSDTGSLPPNLQPGYSPSPTHPPTGQYTLFPLPPTLPTPVVQLHPLLPDPSAPTPTPTSPLLNTSYTHSQNLSQEG